(19) United States
(12) Patent Application Publication
(10) Pub. No.: US 2019/0064165 A1
(43) Pub. Date: Feb. 28, 2019

(54) COMPOSITIONS AND METHODS FOR DETERMINING THE PRESENCE OF ACTIVE LEUKOCYTE CELLS USING AN ELECTROCHEMICAL ASSAY

(71) Applicant: Parvizi Surgical Innovation, LLC, Gladwyne, PA (US)

(72) Inventors: Andrew Neil FLEISCHMAN, Longport, NJ (US); Javad PARVIZI, Gladwyne, PA (US)

(21) Appl. No.: 16/087,411

(22) PCT Filed: Mar. 17, 2017
(86) PCT No.: PCT/US17/22976

§ 371 Date: Sep. 21, 2018

Related U.S. Application Data
(60) Provisional application No. 62/311,405, filed on Mar. 22, 2016.
(60) Provisional application No. 62/352,561, filed on Jun. 21, 2016.

(51) Int. Cl. G01N 33/569; C07C 69/708; G01N 27/327; C07C 229/10; C07K 5/11

(52) U.S. Cl. CPC G01N 33/56972; C07C 69/708; C07K 5/1019; C07C 229/10; G01N 27/3277

(57) ABSTRACT

The present disclosure relates to compositions, methods and test devices for determining the presence of active leukocyte cells, for example, by using novel LE and/or FINE substrates in an electrochemical assay.

### I. CROSS-REFERENCE TO RELATED APPLICATIONS

This application claims priority to U.S. Provisional Patent Application Ser. No. 62/311,405, filed Mar. 22, 2016, and to U.S. Provisional Patent Application Ser. No. 62/352,560, filed Jun. 21, 2016. The disclosure of both references are hereby incorporated by reference in their entirety.

### II. FIELD OF THE INVENTION

The present disclosure relates to a novel application of an electrochemical assay for the determination of the activity of leukocyte cells within a test sample. More particularly, the present disclosure relates to novel methods and kits for determining the activity of enzymes released by active leukocyte cells, especially leukocyte esterase and human neutrophil elastase, in a patient at risk of developing an infection.

### III. BACKGROUND OF THE INVENTION

The presence of an abnormally high number of leukocyte cells in urine is a commonly used indicator of an infectious process. Historically, technicians have relied on manual visual count under a microscope. This visual technique has been largely replaced by a dipstick assay for detection of urogenital infections. In a large majority of such commercial ‘dipstick’ assays, activity of the enzyme leukocyte esterase (“LE”) is used as a proxy for the presence of active leukocyte cells. An assay for human neutrophil elastase (“HNE”) has also been reported to have great sensitivity for the diagnosis of urethral infections in men.

Known assays for LE are chromogenic, in that the presence of enzyme activity is reported based upon a color change. Typically, a color test strip can be matched to a color chart with 3-4 increments of increasing color intensity (from none to 2+/3+), which represents a LE concentration of 30 ng/mL to greater than 1500 ng/mL. However, there are clear disadvantages to a colorimetric assay. With only 3-4 available color intensity increments, resolution of differences in leukocyte esterase concentration may be quite difficult. In addition, inter-rater and even intra-rater reliability in classifying such color increments may be poor. This is especially true for instances in which dipstick results are less definitive (trace or 1+); test results, in such cases, may be too unreliable for making treatment decisions. Thus, the utility of dipstick results is limited to cases in which leukocyte esterase activity is exceedingly high. Any substance that changes the color of urine (e.g. nitrofurantoin, phenazopyridine) also affects dipstick readings.

In recent years, leukocyte esterase testing has piqued the interest of physicians for applications using seous fluid, such as that from joint, lung, abdominal, or even middle ear effusions. While results have been quite promising for the diagnosis of periprosthetic joint infection (PJI), a colorimetric test is rendered impractical in as many as 17-29% of samples due to the presence of blood or debris. The same would be true for other body cavities, for which aspiration often does not yet often always yield clear fluid. Further, a colorimetric leukocyte esterase test cannot be attempted on serum samples.

More recently, a lactate ester substrate has been demonstrated to have improvement in terms of LE assay sensitivity and speed. The alcohol portion is released as a hydroxyl-pyrrole compound, which then reacts with diazonium salt to produce a purple azo dye. However, such an assay has limited utility in bloody or turbid fluid conditions and would require expensive optical sensors to provide a precise, quantitative measurement. Accordingly, there is an urgent need for improved substrates and assays to detect leukocytes and leukocyte enzymes in a sample.

### IV. SUMMARY OF THE INVENTION

In one aspect, the disclosure is directed towards a method for screening, detecting and confirming an infection in patients at risk of an infection or those patients who have already exhibited symptoms associated with an infection. In one embodiment, the method follows the step of obtaining a sample from the subject in need, detecting the presence or absence of leukocyte markers in the sample, and instituting a therapeutic regimen based on the degree and presence of the leukocyte markers in the sample.

In some embodiments, the leukocyte markers can be one or any combinations of such markers as cytokines, chemokines, oxygen and nitrogen radicals, leukocyte elastase, leukocyte esterase, neutrophil elastase, gelatinases, IL-1β, metalloproteinases (MMPs), cathepsins, such as cathepsin A and cathepsin B, phospholipases, such as, for example, phospholipase A and phospholipase B.

In one aspect, the present disclosure is directed to a composition comprising a leukocyte enzyme or specifically a neutrophil enzyme substrate. In some embodiments, the leukocyte enzyme comprises leukocyte esterase (“LE”). In some embodiments, the leukocyte enzyme substrates comprises an LE substrate. In some embodiments, the leukocyte enzyme comprises human neutrophil elastase (“HNE”). In some embodiments, the leukocyte enzyme substrate comprises an FINE substrate. In an alternative embodiment, the composition comprise both an LE substrate and a HNE substrate. In yet another embodiment, the composition may contain additional substrates specific to other enzymes or biomarkders than LE and HNE.

In some embodiments, the substrates is specific for LE or HNE. In some embodiments, the substrates may follow Formula I as depicted below:

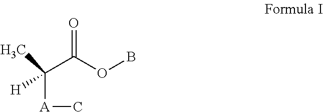

wherein A determines the acyl group at the ester cleavage site, B comprises a moiety capable of participating in a redox reaction, and C comprises an alcohol or amine blocking group. In some embodiments, A comprises an amino group. In some embodiments. A comprises an ether group. In some embodiments, B comprises a redox active alcohol intermediate. In some embodiments, B comprises a quinone. In some embodiments, B comprises a hydroquinone. In some embodiments, B comprises a substituted quinone or a substituted hydroquinone. In some embodiments, C comprises a tosyl protecting group. In some embodiments, the oxygen linking B in Formula I is substituted with an amino group. In further embodiments. B comprises phenylenediamine. In some embodiments, B comprises a substituted phenylenediamine.

In some embodiments, the LE substrate comprises a compound as described in Formula II below:

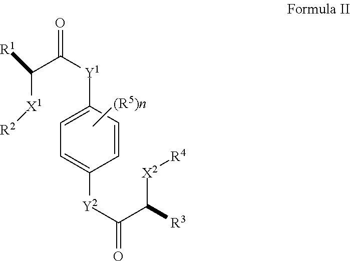

X¹ and X² are independently O, S or NR^{a}, R^{a} is an H, an alkyl or an aryl group. X¹ and X² can be both oxygen or both NR^{a}. Alternatively, one of X¹ and X² is oxygen and the other is NR^{a}.

Y¹ and Y² are independently O or NR^{a}. R^{a} is as described above. Y¹ and Y² can be both oxygen or both NR^{a}. Alternatively, one of Y¹ and Y² is oxygen and the other is NR^{a}.

R¹ and R² are independently an alkyl or an aryl group or a substituted alkyl, a substituted aryl or a protecting group. In some embodiments, R¹ and R² are both methyl. In some embodiments, R¹ and R² may be a tosyl. In some embodiments, R² may be a tosyl.

R³ and R⁴ are independently an alkyl, a protecting group or a peptide moiety. Example of a protecting group includes tosyl, benzoyl, benzyl, trimethylsilyl, [bis-(4-methoxyphenyl)phenylmethyl], carbobenzyloxy, and tert-Butyloxycarbonyl, 9-Fluorenylmethyloxycarbonyl. In one embodiment, R⁴ may be a tosyl. The peptide moiety can include any combination of natural and/or non-natural amino acids.

Each of the R⁵ on the ring is independently a halogen atom; a hydroxyl group; a C₁-C₆ alkyl group; a C₃-C₆ cycloalkyl group; a C₃-C₆ cycloalkyl C₁-C₆ alkyl group; a C₂-C₆ alkenyl group; a C₁-C₆ alkynyl group; a C₁-C₆ haloalkyl group (including trifluoro C₁-C₆alkyl); a C₂-C₆ haloalkenyl group; a C₂-C₆ haloalkynyl group; a C₃-C₆ halocycloalkyl group; a C₃-C₆ halocycloalkyl C₁-C₆ alkyl group; a C₁-C₆ alkoxy group; a C₃-C₆ cycloalkyloxy group; a C₂-C₆ alkenyloxy group; a C₂-C₆ alkynyloxy group; a C₁-C₆ alkylcarbonyloxy group; a C₁-C₆ haloalkoxy group; a C₁-C₆ alkylthio group; a C₁-C₆ alkylsulfinyl group; a C₁-C₆ alkylsulfonyl group; a C₁-C₆ haloalkylthio group; a C₁-C₆ haloalkylsulfinyl group; a C₁-C₆ haloalkylsulfonyl group; an amino group; a C₁-C₆ alkylcarbonylamino group; a mono(C₁-C₆ alkyl)amino group; a di(C₁-C₆ alkyl)amino group; a hydroxy C₁-C₆ alkyl group; a C₁-C₆ alkoxy C₁-C₄ alkyl group; a C₁-C₆ alkylthio C₁-C₆ alkyl group; a C₁-C₆ alkylsulfinyl C₁-C₆ alkyl group; a C₁-C₆ alkylsulfonyl C₁-C₆ alkyl group; a C₁-C₆ haloalkylthio C₁-C₆ alkyl group; a C₁-C₆ haloalkylsulfinyl C₁-C₆ alkyl group; a C₁-C₆ haloalkylsulfonyl C₁-C₆ alkyl group; a cyano C₁-C₆ alkyl group, a C₁-C₆ alkoxy C₁-C₆ alkoxy group; a C₃-C₆ cycloalkyl C₁-C₆ alkyloxy group; a C₁-C₆ haloalkoxy C₁-C₆ alkoxy group; a cyano C₁-C₆ alkoxy group; a C₁-C₆ acyl group; a C₁-C₆ alkoxyimino C₁-C₆ alkyl group; a carboxyl group; a C₁-C₆ alkoxycarbonyl group; a carbamoyl group; a mono(C₁-C₆ alkyl)aminocarbonyl group; a di(C₁-C₆ alkyl)aminocarbonyl group; a nitro group; or a cyano group. n is 0, 1, 2, 3, or 4.

In some embodiments, the LE substrate comprises 4-((tosyl-L-alanyl)oxy)phenyl tosyl-L-alaninate. In some embodiments, the LE substrate comprises 4-(((S)-2-(tosyloxy)propanoyl)oxy)phenyl (S)-2-(tosyloxy)propanoate. In some embodiments, the LE substrate comprises a phenylenediamine variant of one of 4-((tosyl-L-alanyl)oxy)phenyl tosyl-L-alaninate and 4-(((S)-2-(tosyloxy)propanoyl)oxy)phenyl (S)-2-(tosyloxy)propanoate.

In some embodiments, the HNE substrate comprises a compound as described in Formula III below:

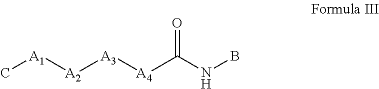

wherein A₁-A₂-A₃-A₄ represent a core tetrapeptide scaffold sequence which serves as the enzyme active site, B comprises a moiety capable of participating in a redox reaction, and C comprises an acyl group. In some embodiments, A₁-A₂-A₃-A₄ comprise AAPV (SEQ ID NO: I). In some embodiments, SEQ ID NO: 1 has conservative substitutions. In some embodiments, B comprises a redox active alcohol intermediate. In some embodiments, B comprises a derivative of phenol. B comprises a quinone. In some embodiments, B comprises a hydroquinone. In some embodiments, B comprises a substituted quinone or a substituted hydroquinone. In some embodiments, C comprises N-methyoxysuccinyl.

In some embodiments, the HNE substrate comprises 3-{[(1S)-1-{[(2S)-1-(5-{[(1S)-1-({4-[(2S)-2-({1-[(2S)-2-[(2S)-2-(3-carboxypropanamido)propanamido]propanoyl]pyrrolidin-2-yl}formamido)-3-methylbutanamido]phenyl}carbamoyl)-2-methylpropyl]carbamoyl}imidazolidin-1-yl)-1-oxopropan-2-yl]carbamoyl}ethyl]carbamoyl}propanoic acid.

In some embodiments, the leukocyte enzyme substrate is included in an assay. In some embodiments, the assay comprises an electrochemical assay. In an alternative embodiment, the assay may include a colorimetric step in combination with the electrochemical assay. In some embodiments, the electrochemical assay comprises an internally calibrated electrochemical continuous enzyme assay (“ICECEA”). In some embodiments, the electrochemical assay comprises a leukocyte substrate of the present disclosure and an electrochemical measuring device. In some embodiments, the electrochemical measuring device includes a working electrode, a reference electrode, and an auxiliary electrode.

In some embodiments, the present disclosure is directed to a method of detecting the presence of a leukocyte enzyme in a sample and instituting a therapeutic plan. In some embodiments, the presence of a leukocyte enzyme in the sample indicates the presence of a leukocyte in the sample. In some embodiments, the leukocyte enzyme comprises LE. In some embodiments, the leukocyte enzyme comprises human neutrophil elastase HNE. In some embodiments, the leukocyte enzyme is detected by contacting the enzyme with a substrate of the enzyme. In some embodiments, the substrate is any LE substrate of the present disclosure. In some embodiments, the substrate is any FINE substrate of the present disclosure.

In some embodiments, the amount of leukocyte enzyme present in the sample is quantified. In some embodiments, the presence of a leukocyte in the sample is indicative of an infection. In some embodiments, the infection comprises a urinary tract infection (“UTI”). In some embodiments, the infection comprises a periprosthetic joint infection (“PJI”). In some embodiments, the infection comprises spontaneous bacterial peritonitis (“SBP”). In some embodiments, the sample comprises a biological sample. In some embodiments, the biological sample comprises one of urine, sputum, synovial fluid, pleural fluid, pericardial fluid, peritoneal fluid, cerebrospinal fluid (“CSF”) and middle ear fluid.

In some embodiments, the method of screening a patient at risk of developing an infection following the steps of detecting the presence of a leukocyte enzyme in a sample by contacting a leukocyte enzyme with a substrate in an assay. In some embodiments, the assay comprises an electrochemical assay. In some embodiments, the electrochemical assay comprises an internally calibrated electrochemical continuous enzyme assay (“ICECEA”).

In some embodiments, the method of detecting the presence of a leukocyte enzyme in an electrochemical assay comprises a step of adding a first aliquot of a reactant or product of a leukocyte enzyme to a substrate of the leukocyte enzyme. In some embodiments, the leukocyte enzyme substrate is in an electrolyte solution. In some embodiments, the method comprises a step of measuring current flowing through an electrode of the electrochemical assay. In some embodiments, the method comprises a step of adding at least one additional aliquot of the reactant or product of a leukocyte enzyme to the substrate of the leukocyte enzyme. In some embodiments, the method comprises a step of measuring current flowing through an electrode of the electrochemical assay for a second time. In some embodiments, the method comprises a step of adding the leukocyte enzyme to the substrate of the leukocyte enzyme. In some embodiments, the method comprises a step of measuring current flowing through an electrode of the electrochemical assay for a third time.

In some embodiments, the method of screening a patient for infection by detecting the presence of a leukocyte enzyme in an electrochemical assay following a process including a step of adding a first aliquot of a leukocyte enzyme to a substrate of the leukocyte enzyme. In some embodiments, the leukocyte enzyme substrate is in an electrolyte solution. In some embodiments, the method comprises a step of measuring current flowing through an electrode of the electrochemical assay. In some embodiments, the method comprises a step of adding at least one additional aliquot of the leukocyte enzyme to the substrate of the leukocyte enzyme. In some embodiments, the method comprises a step of measuring current flowing through an electrode of the electrochemical assay for a second time. In some embodiments, the method comprises a step of adding a product or reactant of a leukocyte enzyme to the substrate of the leukocyte enzyme. In some embodiments, the method comprises a step of measuring current flowing through an electrode of the electrochemical assay for a third time.

In another aspect, the present disclosure is directed to kits containing suitable substrate, direction for optimizing the results and optionally providing patient specific therapeutic regimen based on the observed results.

### V. BRIEF DESCRIPTION OF THE FIGURES

FIG. 1 represents an initial hydroquinone substrate and first ester hydrolysis step.

FIG. 2 represents a semiquinone intermediate and second ester hydrolysis step.

FIG. 3 represents a final benzoquinone oxidation product.

FIG. 4 represents the results of using 4-((tosyl-L-alanyl)oxy)phenyl tosyl-L-alaninate (“TAPTA”) in an internally calibrated electrochemical continuous enzyme assay (ICECEA).

FIG. 5 represents the NMR of 4-((tosyl-L-alanyl)oxy)phenyl tosyl-L-alaninate (“TAPTA”).

### VI. DETAILED DESCRIPTION OF THE INVENTION

As used herein and in the appended claims, the singular forms “a”, “and” and “the” include plural references unless the context clearly dictates otherwise.

As used herein, “leukocyte” may refer to any white blood cell (“WBC”). Leukocytes are cells of the immune system that are involved in protecting the body against infectious disease and invading pathogens. All leukocytes/WBCs are divided into five classes based on morphological characteristics that differentiate themselves from one another. They include neutrophils, eosinophils, basophils, monocytes, and lymphocytes. Neutrophils comprise approximately 40-75% of leukocytes, eosinophils comprise approximately 1-6% of leukocytes, basophils comprise less than 1% of leukocytes, monocytes comprise approximately 2-10% of leukocytes, and lymphocytes (e.g. B lymphocytes and T lymphocytes) comprise approximately 20-45% of leukocytes.

The term “patient” as used herein may refer to a biological system to which a treatment can be administered. A biological system can include, for example, an individual cell, a set of cells (e.g. a cell culture), an organ, a tissue, or multi-cellular organism. A “patient” can refer to a human patient or a non-human patient. In preferred embodiments, the patient is a human patient.

The terms “effective amount” or “therapeutically effective amount” as used herein may refer to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing as well as after their appearance. Thus, “treating” or “treatment” includes “preventing” or “prevention” of disease. The terms “prevent” or “preventing” refer to prophylactic and/or preventative measures, wherein the object is to prevent or slow down the targeted pathologic condition or disorder.

The present disclosure relates to compositions and methods for rapid detection (including determining the relative activity) of enzymes released by active leukocyte cells, e.g. leukocyte enzymes released by active leukocyte cells, in particular leukocyte esterase (“LE”) and human neutrophil elastase (“HNE”).

In at least one aspect of the present disclosure, a method of screening a subject for infection is described, said method comprising the steps of (a) obtaining a sample of tissue or bodily fluid from a subject at risk of developing an infection, (b) applying the sample to a detector device, wherein the detector device comprises at least one substrate which is specific for at least one of LE and/or HNE, wherein said at least one substrate is adapted to detect a threshold level at least one of LE and/or HNE, said threshold level correlated with a presence of infection; (c) ascertaining the threshold levels of LE and/or fINE present in said sample, wherein if the concentration each of LE and/or HNE exceeds the threshold level, and further wherein such measurement is a positive screen for infection.

The disclosure provides a method wherein the infection is a periprosthetic joint infection (PJI). In some embodiments, the threshold level of leukocyte esterase (LE) for detection of PJI is at least about 20 pg/ml of leukocyte esterase in a synovial fluid sample.

The compositions and methods for rapid detection utilize specific substrates for detecting leukocyte enzymes, e.g. LE and HNE, referred to as LE substrates and HNE substrates respectively. The compositions and methods for rapid detection may utilize electrochemical assays to detect the leukocyte enzymes, in particular, internally calibrated electrochemical continuous enzyme assay (“ICECEA”), but are not necessarily limited as such.

In some embodiments, the substrates are capable of detecting LE. Such substrates are readily hydrolyzed by LE to generate a redox intermediate, which can provide a detectable electrochemical response. In some embodiments, the substrates for detecting LE (i.e. “LE substrates”) may follow Formula I as depicted below:

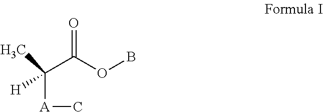

Where A determines the acyl group, e.g. an alanine or lactate, at the ester cleavage site with enzyme specificity for leukocyte esterase and B is a moiety capable of participating in a redox reaction, which can be detected using an electrochemical assay (e.g. by using ICECEA). The acyl group A is protected using any effective amine or alcohol blocking group C (e.g. a tosyl group). The alcohol intermediate of the ester, moiety B, to be released upon hydrolysis by the esterase is a redox substrate, and participates in a redox reaction. Additionally, the oxygen linking B in Formula I may be substituted with an —NH linking moiety (i.e. the ester group presented in Formula I may be substituted with an amido group) and still be within the scope of the present disclosure.

The amine or alcohol blocking group C may comprise any of the following: acetyl (Ac), benzoyl (Bz), benzyl (Bn), β-methoxyethoxymethyl ether (MEM), dimethoxytrityl (DMT), methyoxymethyl ether (MOM), methoxytrityl [(4-methoxyphenyl)diphenylmethyl](MMT), p-Methoxybenzyl ether (PMB), methylthiomethyl ether, pivaloyl (Piv), tetrahydropyranyl (THP), tetrahydrofuran (THF), trityl (Tr), silyl ethers (e.g. TMS, TBDMS, TOM, TIPS), methyl ethers, and ethoxyethyl ethers (EE), carbobenzyloxy (Cbz); p-methoxybenzyl carbonyl (Moz or McOZ), tert-butyloxycarbonyl (BOC), 9-fluorenylmethyloxycarbonyl (FMOC), carbamate, 3,4-Dimethoxybenzyl (DMPM), p-methoxyphenyl (PMP), tosyl (Ts), trichloroethyl chloroformate (Troc), and other sulfonamaides (e.g.Nosyl and Nps).

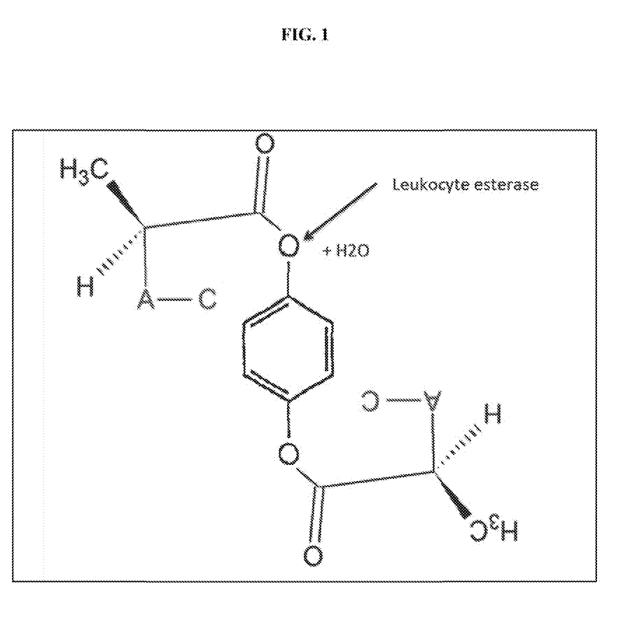

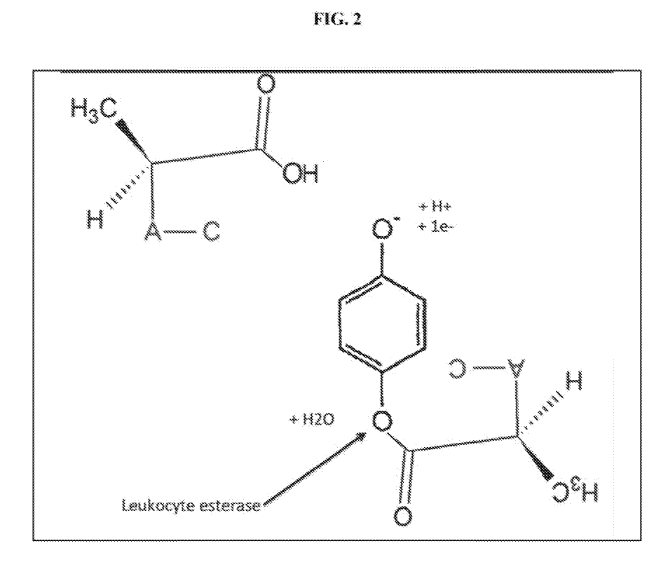

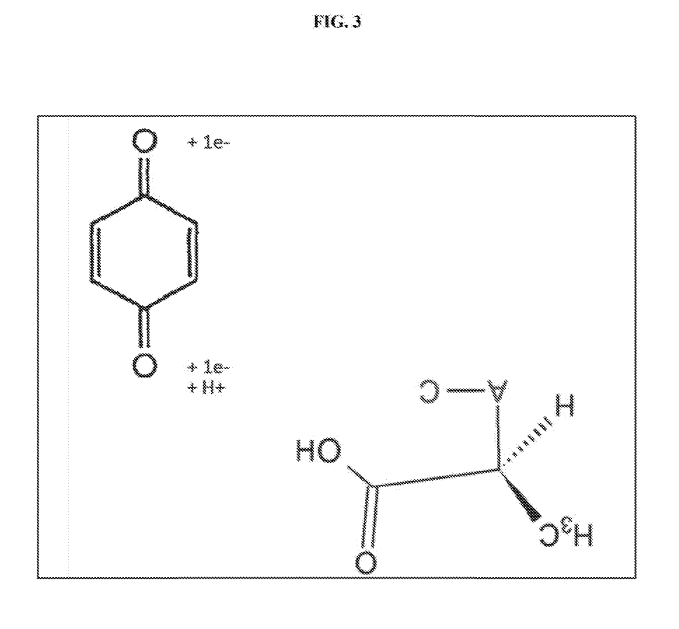

In some embodiments, the redox moiety B is a derivate of phenol, which may form an ester through its hydroxyl group. Such an intermediate may undergo oxidation to release an electron. For example, but not necessarily limited to, one phenol derivative, hydroquinone, contains two hydroxyl groups in a para conformation. Each hydroxyl group can be bound to form a distinct lactate ester, which is independently a substrate of leukocyte esterase (FIG. 1). The resulting duplex substrate has two potential target sites for leukocyte esterase activity, and breakdown of the substrate is stepwise. Ester hydrolysis with leukocyte esterase at the first target will occur relatively slow due to molecular hindrance of the active sites; however, subsequent hydrolysis of the second active site will occur more quickly. This may effectively improve the specificity of an electrochemical assay, as non-specific hydrolysis would be less likely to begin the cascade. After the first ester hydrolysis step, an oxidation reaction can release an electron with removal of a hydrogen atom forming a semiquinone lactate ester intermediate (FIG. 2). After subsequent hydrolysis of the remaining ester, the quinone-based intermediate is released and can be further oxidized to form para-benzoquine. Para-benzoquine is reduced at low potentials, which minimizes interference from other redox active species within the sample and may improve assay selectivity. The final product is shown in FIG. 3.

In other aspects, methods of treating a patient with positive indication of LE and HNE is described. In one embodiment, the serious infections caused by Gram-positive bacteria are currently difficult to treat because many of these pathogens are now resistant to standard antimicrobial agents. To that end, at least one aspect of the disclosure is to prophylactically treat a patient prior to any invasive operation to minimize risk of infection. In at least one embodiment, patients identified as suffering from an infection may be initiated a comprehensive treatment plan including administering antimicrobial agent, such as pennicilians, cephlosporins, tetracyclines, daptomycin, tigecycline, linezolid, quinupristin/dalfopristin and dalbavancin and the like that may be useful in combating an active infection. In other embodiments, methods of screening or detecting risk of PJI, by developing useful for the treatment of infections due to drug-resistant Gram-positives and Gram-negatives.

In some embodiments, B comprises a quinone. In some embodiments, B comprises a hydroquinone. In some embodiments, B comprises a substituted quinone or a substituted hydroquinone. In some embodiments, C comprises a tosyl protecting group. In some embodiments, the oxygen linking B in Formula II is substituted with an amino group. In further embodiments, B comprises phenylenediamine. In some embodiments, B comprises substituted phenylenediamine.

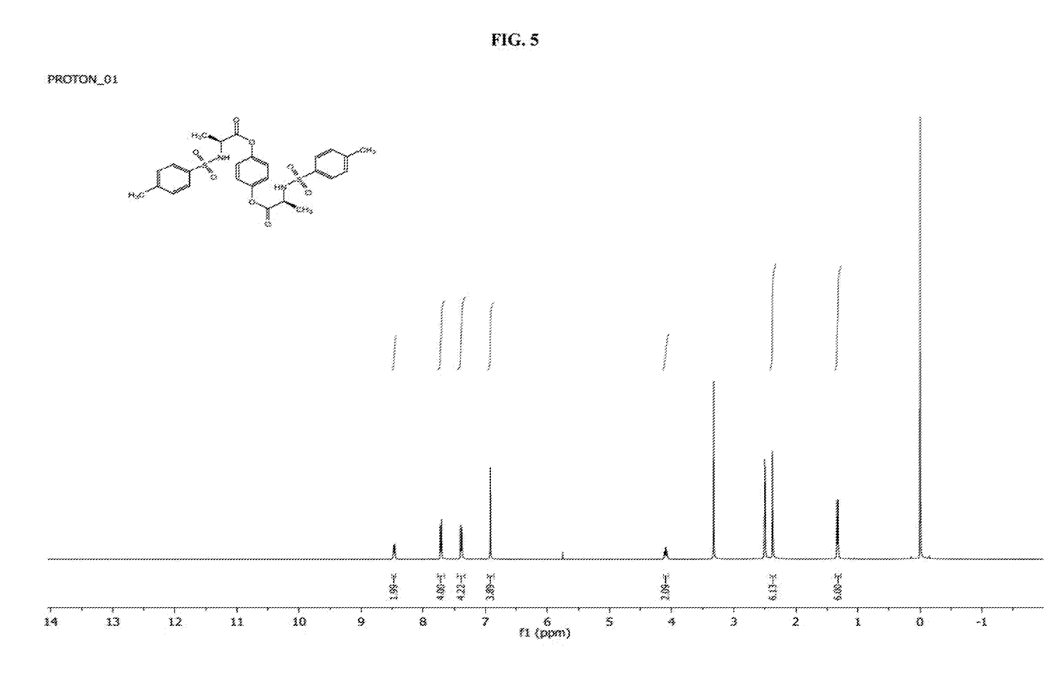

Two specific, explicitly non-limiting examples of substrates for detecting leukocyte esterase (“LE”) that are within the scope of Formula I include 4-((tosyl-L-alanyl)oxy)phenyl tosyl-L-alaninate (Compound A below) and 4-(((S)-2-(tosyloxy)propanoyl)oxy)phenyl (S)-2-(tosyloxy)propanoate (Compound B below). Compound A is also referred to herein as “TAPTA.” An NMR of Compound A is shown in FIG. 5, illustrating the tosyl moiety structure and its attachment. Phenylethylenediamine variants of Compound A and Compound B (i.e. the para-oxygens are replaced with NH linkers) are also to be considered within the scope of the present disclosure and are likewise suitable for inclusion in electrochemical assays of the present disclosure (e.g. in ICECEA).

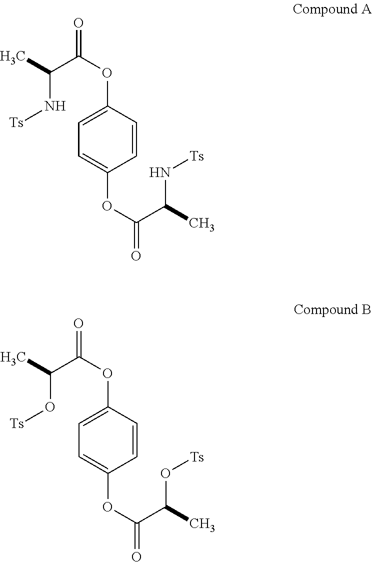 [SMILES: Cc1ccc(S(=O)(=O)N[C@@H](C)C(=O)Oc2ccc(OC(=O)[C@H](C)NS(=O)(=O)c3ccc(C)cc3)cc2)cc1 ; Cc1ccc(S(=O)(=O)O[C@@H](C)C(=O)Oc2ccc(OC(=O)[C@H](C)OS(=O)(=O)c3ccc(C)cc3)cc2)cc1]

In some embodiments, the LE substrate comprises a composition as described in Formula ii below:

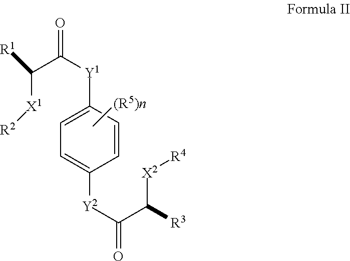

X¹ and X² are independently O, S or NR^{a}. R^{a} is an H, an alkyl or an aryl group. X^{L} and X² can be both oxygen or both NR^{a}. Alternatively, one of X¹ and X² is oxygen and the other is NR^{a}.

Y¹ and Y² are independently O, S or NR^{a}. R^{a} is as described above. Y¹ and Y² can be both oxygen or both NR³. Alternatively, one of Y¹ and Y² is oxygen and the other is NR^{a}.

R¹ and R² are independently an alkyl or an aryl group or a substituted alkyl, a substituted aryl or a protecting group. In some embodiments, R¹ or R² or both is methyl. In some embodiments, R¹ or R² or both may be a tosyl. In one embodiment, R² is a tosyl.

R³ and R⁴ are independently an alkyl, a protecting group such as tosyl, benzoyl, benzyl, trimethylsilyl, [bis-(4-methoxyphenyl)phenylmethyl], carbobenzyloxy, tert-Butyloxycarbonyl, 9-Fluorenylmethyloxycarbonyl, or a peptide moiety. In one embodiment, R⁴ is a tosyl. The peptide moiety can include any combination of natural and/or non-natural amino acids.

R² and R⁴ may also comprise any of the following: acetyl (Ac), benzoyl (Bz), benzyl (Bn), β-methoxyethoxymethyl ether (MEM), dimethoxytrityl (DMT), methyoxymethyl ether (MOM), methoxytrityl [(4-methoxyphenyl)diphenylmethyl] (MMT), p-Methoxybenzyl ether (PMB), methylthiomethyl ether, pivaloyl (Piv), tetrahydropyranyl (THP), tetrahydrofuran THrF), trityl (Tr), silyl ethers (e.g. TMS, TBDMS, TOM, TIPS), methyl ethers, and ethoxyethyl ethers (EE), carbobenzyloxy (Cbz); p-methoxybenzyl carbonyl (Moz or MeOZ), tert-butyloxycarbonyl (BOC), 9-fluorenylmethyloxycarbonyl (FMOC), carbamate, 3,4-Dimethoxybenzyl (DMPM), p-methoxyphenyl (PMP), tosyl (l's), trichloroethyl chloroformate (Troc), and other sulfonamaides (e.g.Nosyl and Nps). In one embodiment, protecting group can be any one of tosyl, benzoyl, benzyl, trimethylsilyl, [bis-(4-methoxyphenyl)phenylmethyl], carbobenzyloxy, tert-Butyloxycarbonyl, 9-Fluorenylmethyloxycarbonyl.

Each of the R⁵ on the ring is independently a halogen atom; a hydroxyl group; a C₁-C₆ alkyl group; a C₃-C₆ cycloalkyl group; a C₃-C₆ cycloalkyl C₁-C₆ alkyl group; a C₂-C₆ alkenyl group; a C₂-C₆ alkynyl group; a C₁-C₆ haloalkyl group (including trifluoro C₁-C₆alkyl); a C₂-C₆ haloalkenyl group; a C₂-C₆ haloalkynyl group; a C₃-C₆ halocycloalkyl group; a C₃-C₆ halocycloalkyl C₁-C₆ alkyl group; a C₁-C₆ alkoxy group; a C₃-C₆ cycloalkyloxy group; a C₂-C₆ alkenyloxy group; a C₂-C₆ alkynyloxy group; a C₁-C₆ alkylcarbonyloxy group; a C₁-C₆ haloalkoxy group; a C₁-C₆ alkylthio group; a C₁-C₆ alkylsulfinyl group; a C₁-C₆ alkylsulfonyl group; a C₁-C₆ haloalkylthio group; a C₁-C₆ haloalkylsulfinyl group; a C₁-C₆ haloalkylsulfonyl group; an amino group; a C₁-C₆ alkylcarbonylamino group; a mono(C₁-C₆ alkyl)amino group; a di(C₁-C₆ alkyl)amino group; a hydroxy C₁-C₆ alkyl group; a C₁-C₆ alkoxy C₁-C₆ alkyl group; a C₁-C₆ alkylthio C₁-C₆ alkyl group; a C₁-C₆ alkylsulfinyl C₁-C₆ alkyl group; a C₁-C₆ alkylsulfonyl C₁-C₆ alkyl group; a C₁-C₆ haloalkylthio C₁-C₆ alkyl group; a C₁-C₆ haloalkylsulfinyl C₁-C₆ alkyl group; a C₁-C₆ haloalkylsulfonyl C₁-C₆ alkyl group; a cyano C₁-C₆ alkyl group; a C₁-C₆ alkoxy C₁-C₆ alkoxy group; a C₃-C₆ cycloalkyl C₁-C₆ alkyloxy group; a C₁-C₆ haloalkoxy C₁-C₆ alkoxy group; a cyano C₁-C₆ alkoxy group; a C₁-C₆ acyl group; a C₁-C₆ alkoxyimino C₁-C₆ alkyl group; a carboxyl group; a C₁-C₆ alkoxycarbonyl group; a carbamoyl group; a mono(C₁-C₆ alkyl)aminocarbonyl group; a di(C₁-C₆ alkyl)aminocarbonyl group; a nitro group; or a cyano group. n is 0, 1, 2, 3, or 4. In at least one embodiment, X¹ and X² are independently O or NR^{a}. R^{a} is an H, an alkyl or an aryl group. X¹ and X² can be both oxygen or both NR^{a}. Alternatively, one of X¹ and X² is oxygen and the other is NR^{a}. in yet another embodiment, Y¹ and Y² are independently O, or NR^{a}.

In some embodiments, the substrates detect human neutrophil elastase (“HNE”). In some embodiments, the substrates for detecting HNE (i.e. “HNE substrates”) may follow Formula III as depicted below:

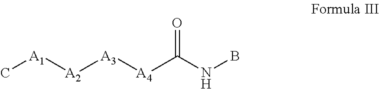

A₁ through A₄ (i.e. A₁-A₂-A₃-A₄) represent a core tetrapeptide scaffold sequence, which serves as the enzyme active site (i.e. the active site for human neutrophil elastase/HNE). A tetrapeptide sequence of Ala-Ala-Pro-Val (AAPV) (SEQ II) NO: I) is most common, but natural or unnatural amino acids may be substituted at any of the four peptide sites in order to improve substrate sensitivity for HNE. For example, conservative substitutions may be made for SEQ ID NO: 1 and still be within the scope of the present disclosure. As used herein, “conservative substitutions” are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

B in Formula III represents a redox moiety, similar to the LE substrate displayed in Formula I above. For example, B may comprise derivate of phenol, which may form an ester through its hydroxyl group, e.g., a redox active alcohol intermediate. This may comprise, for example, a hydroquinone intermediate or hydroquinone-based redox groups. C in Formula IIO represents an acyl group, for example, N-methyoxysuccinyl. The acyl group may serve to improve substrate sensitivity for HNE, and some acyl groups, for example N-methoxysuccinyl, may also increase substrate solubility.

One specific, explicitly non-limiting example of a substrate for detecting HNE that is within the scope of Formula III includes 3-{[(1S)-1-{[(2S)-1-(5-{[(1S)-1-({4-[(2S)-2-({1-[(2S)-2-[(2S)-2-(3-carboxypropanamido)propanamido]propanoyl]pyrrolidin-2-yl}formamido)-3-methylbutanamido]phenyl}carbamoyl)-2-methylpropyl]carbamoyl}imidazolidin-1-yl)-1-oxopropan-2-yl]carbamoyl}ethyl]carbamoyl}propanoic acid, Compound C below.

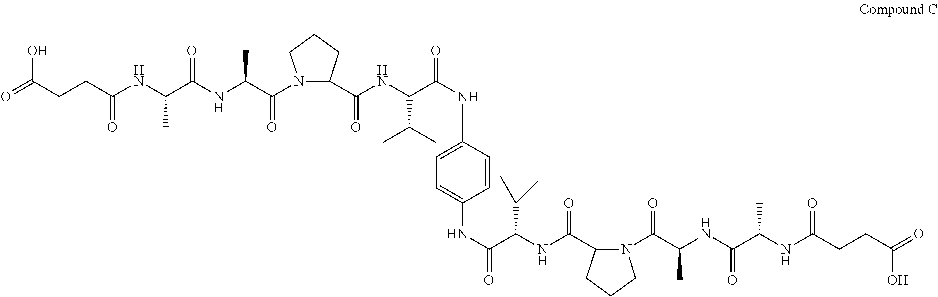

As described herein, leukocytes are capable of producing leukocyte enzymes that are able to be detected and/or quantified by the electrochemical assays (i.e. ICECEA) of the present disclosure.

Leukocyte enzymes may include, for example, those described in WO 2010/036930, hereby incorporated by reference in its entirety, such as, for example, IL-1β, leukocyte elastase, leukocyte esterase, and/or gelatinase B, along with human neutrophil elastase.

Leukocyte esterase (“LE”) is an esterase produced by leukocytes (white blood cells). LE is the subject of, for example, urine tests for the presence of leukocytes/WBCs and other abnormalities associated with infection. Human neutrophil elastase (“HNE”), also known as human leukocyte elastase (“HLE”), is a serine protease. It is in the same family as chymotrypsin and possesses broad substrate activity. HNE is secreted by neutrophils and macrophages, two of the five classes of leukocytes as described herein. HNE is 218 amino acids long and has two asparagine-linked carbohydrate chains. There are two forms of HNE, deemed IIa and IIb.

The term “sample” as used herein may refer to a biological sample, including a sample of biological tissue or fluid origin obtained in vivo or in vitro. Biological samples can be, but are not limited to, body fluid (e.g., serous fluid, blood, blood plasma, serum, or urine), organs, tissues, fractions, and cells isolated from mammals including, for example, humans. Biological samples also may include sections of the biological sample including tissues. Biological samples may also include extracts from a biological sample, for example, a biological fluid (e.g., blood, serum, peritoneal fluid, and/or urine). Of particular interest, but explicitly non-limiting, are urine, sputum (for example, in a patient diagnosed with cystic fibrosis), peritoneal fluid (for example, in a patient with liver cirrhosis and ascites) and other serous fluids, including but not limited to, for example, synovial fluid, pleural fluid, pericardial fluid, cerebrospinal fluid (“CSF”) and middle ear fluid.

In some embodiments, the presence of leukocytes, i.e. as determined by detecting and/or quantifying the amount of a leukocyte enzyme (e.g. LE and/or HNE) present in said biological sample may indicate the presence of an infection in a subject. Such embodiments may utilize the LE and/or HNE substrates of the present disclosure in an electrochemical assay, in particular ICECEA as described herein. For example, the presence of LE and/or HNE in urine may indicate a subject as having a urinary tract infection (“UTI”). Similarly, the presence of LE and/or HNE in synovial fluid may indicate a subject as having a joint infection, for example but not necessarily limited to a periprosthetic joint infection (“PJI”). These examples of indicating the presence of infection are not limited as such, as these are merely exemplary uses of the substrates of the present disclosure, and they may or may not be utilized in an electrochemical assay, for example, in an ICECEA.

In some embodiments, the substrates of the present disclosure are used to indicate a subject as having periprosthetic joint infection (PJI). PJI is a devastating complication fobllowing total joint arthroplasty, which remains a challenge for surgeons both diagnostically and therapeutically. Establishing an accurate and timely diagnosis of PJI is of critical importance for making treatment decisions. For patients presenting with a painful prosthesis, it is important to complete a work-up to either rule out or diagnose the presence of infection. In most cases, serological testing, including erythrocyte sedimentation rate (ESR) and C-reactive protein (CRP), is the initial screening test of choice. In patients with elevated serological markers or even just a high suspicion of infection, the next step is to perform joint aspiration for testing of synovial fluid. Classically, bacterial culture of synovial fluid has been used to make the diagnosis of PJI. As bacterial culture is not in itself sufficiently sensitive, with as many as 30% of infections being culture negative, orthopedic surgeons also consider the results of serological testing, synovial fluid white blood cell count and polymorphonuclear percentage, and histological analysis to make a diagnosis. Unfortunately, bacterial culture and traditional synovial fluid testing can require days to more than a week to yield a result.

Thus, in some embodiments, synovial fluid aspirated from a painful joint would be tested for LE and/or HNE activity using an enzyme substrate of the present disclosure. For example, this may be accomplished through use of an ICECEA assay as described herein. In such embodiments, the activity of LE and/or HNE would be reported as a continuous measurement of absolute concentration. This could be performed in the office or operating room to yield a result in minutes for point-of-care decision-making.

Based on an accumulation of population data, the level of LE and/or HNE activity can be combined with additional metrics to predict the likelihood that an infection is present. Additional metrics may include the type of joint, a history of prior infection, and the results of serological testing (ESR and CRP). Surgeons can consider the likelihood that an infection is present to determine the most appropriate treatment algorithm for their patient. In cases with a high likelihood that infection is present, treatment for PJI, such as prosthesis extraction and antibiotic spacer placement, incision and debridement, or long-term antibiotic suppression, could be considered based on the acuity of the infection, among other factors. In cases in which there is a moderate likelihood that infection is present, a surgeon could consider initiating treatment or waiting for additional diagnostic results. Finally, other etiologies for a painful prosthesis may be considered in cases for which the likelihood of the presence of infection is low or for which infection has largely been ruled out.

In addition to making an initial diagnosis of infection, the substrates of the current disclosure, e.g. as used in an assay (such as, for example, an ICECEA) may be used to establish the resolution of PJI in order to determine the correct timing for re-implantation of a new prosthesis. The level of LE and/or HNE activity may be used in addition to serological markers and other synovial fluid tests to determine the success of treatment, such as discussed supra. For patients with a persistently elevated LE and/or HNE, surgeons may elect to continue intravenous antibiotics or attempt an exchange of the antibiotic spacer to improve prospects of complete resolution of infection.

In some embodiments, the substrates of the present disclosure are used to indicate a subject as having spontaneous bacterial peritonitis (SBP). SBP is a serious and life threatening complication that is relativity common in patients with liver cirrhosis and ascites. For patients with this complication, a rapid diagnosis and early administration of antibiotics is critical for survival, and in-hospital mortality can be as high as 20%. For patients with ascites, presenting symptoms of fever, change in mental status, and abdominal tenderness are frequent signs of SBP. In such cases, a diagnostic paracentesis is performed and a diagnosis is made based on an absolute neutrophil count above 250 cells/mm³ and/or bacterial culture.

Thus, in some embodiments, ascitic fluid obtained from diagnostic paracentesis would be tested for LE and/or HNE activity using an enzyme substrate of the present disclosure. For example, this may be accomplished through use of an ICECEA assay as described herein. Using an ICECEA assay, the activity of LE or HNE would be reported as a continuous measurement of absolute concentration. Based on an accumulation of population data collected from many patients, the absolute concentration of LE and/or HNE would be compared to gold standard diagnostic criteria to provide a calculation of the probability that SBP is present. The likelihood of infection can be used to inform the treating physician as to the most appropriate treatment algorithm. The measured level of LE or HNE could also provide important prognostic information, with a higher level indicating a worse prognosis.

In some embodiments, the substrates of the present disclosure are used to indicate a subject as having a urinary tract infection (UTI), also known as a urogenital infection. For healthy women with classic UTI symptoms, such as dysuria and frequency, and no vaginal discharge or irritation, a diagnosis of UTI can typically be made on clinical symptoms alone. On the contrary, women with poorly defined symptoms, asymptomatic pregnant females, elderly patients, and children have a much lower pre-test probability for UTI. The present disclosure is not limited to testing women for UTI. The gold standard for diagnosis of UTI is mid-stream urine culture (with >10³-10⁵ organisms) or pyuria (greater than 10⁴ leukocytes per ml).

Thus, in some embodiments, mid-stream urine for symptomatic patients would be tested for leukocyte esterase (“LE”) and/or human neutrophil elastase (“HNE”) activity using an enzyme substrate of the present disclosure. For example, this may be accomplished through use of an ICECEA assay as described herein. Based on population data, likelihood of infection can be determined based on both measurement of LE and/or HNE activity and additional factors, such as the presence of specific symptoms and patient characteristics (i.e. age, gender, pregnancy). Depending on the likelihood of infection, a physician can decide whether or not to administer oral antibiotics.

Population data for the clinical applications of the present disclosure (i.e. in indicating a patient as having an infection, for example, but not limited to, PJI, SBP, and/or UTI) can be used to convert the measure of LE and/or HNE activity to a predictive probability for the presence of infection. The test device itself can be used as a medium to both collect and distribute such population-based data. For example, a smartphone (or similar device) connected electrochemical biosensor can allow physicians to provide selected information to a centralized database, which may then be used to continuously improve the calculation of infection likelihood. The biosensor may also report back to surgeons the likelihood of infection for their individual patient based upon LE and/or HNE activity and additional metrics that can be used to hone their treatment algorithm.

In some embodiments, the substrates for detecting leukocyte enzymes, e.g. LE and/or HNE substrates, are incorporated into an assay. Such an assay may comprise, for example, an electrochemical assay. Electrochemical assays are cost-effective, highly sensitive, and simplify the calibration process. Furthermore, such methods would be just as effective in bloody or turbid fluid. A preferred electrochemical assay comprises an internally calibrated electrochemical continuous enzyme assay (“ICECEA”). Use of a LE substrate of the present disclosure (“TAPTA”) in an ICECEA is described in Example 1, infra. ICECEAs are generally disclosed in PCT/US2014/03713 and U.S. 2016/0040209, the disclosure of which is hereby incorporated in its entirety. ICECEA utilizes integration of an enzyme-free pre-assay calibration with an electrochemical enzyme assay in a continuous experiment. This is believed to result in a uniquely shaped amperometric trace that allows for selective and sensitive determination of enzymes, e.g. LE and HNE, present in a sample.

ICECEAs generally follow the following method as described in U.S. 2016/0040209. First, an enzyme substrate (e.g. an LE and/or HNE substrate of the present disclosure) is placed in a background electrolyte. Next, a reactant or product of an enzymatic reaction of the enzyme is added to the first enzyme substrate/background electrolyte, which creates what is described as a “first assay mixture.” Current flowing through an electrode of the electrochemical assay is then measured after the first assay mixture is formed. Next, the enzyme (e.g. LE and/or HNE) is added to the “first assay mixture” to create a “second assay mixture,” and the current is measured again over a predetermined time period. Enzyme activity is determined based on the change in current over time caused by the addition of the enzyme. While optimally the enzyme is added after the reactant/product is added to the enzyme substrate, the order can be switched, i.e. the enzyme is added to the substrate first and then the reactant/product is added.

The ICECEA includes an electrochemical measuring device. The electrochemical measuring device includes a working electrode, a reference electrode, and an auxiliary electrode. The current is measured through the working electrode. The working electrode may be a noble metal electrode, metal oxide electrode, an electrode made of a carbon allotrope, or a modified electrode. The auxiliary electrode may be a platinum wire. The reference electrode may be Ag/AgCl/NaCl or any other reference electrode. The electrochemical assay system can also be made of only a working electrode and a reference electrode. Measuring the changes in current may be done by collecting an amperometric trace of the current.

Generally, in an ICECEA, adding the reactant/product to the enzyme substrate (in electrolyte) in the electrochemical assay system includes the following steps. First, a first aliquot of the reactant/product is added to the enzyme substrate (in electrolyte). Current flowing through an electrode of the electrochemical assay system is measured after the first aliquot is added. One or more additional aliquots of the reactant/product are added to the mixture and current flowing through an electrode of the electrochemical assay system is measured again. Preferably, at least three aliquots of the reactant/product are added to the enzyme substrate (in electrolyte) before the enzyme is added to the mixture. Alternatively, the aliquots of the reactant/product are added to the substrate (in electrolyte) after the enzyme is added to the mixture.

The enzymatic activity of the enzyme may be determined from the slope of a line created from measuring the current flowing through a working electrode of the electrochemical assay system after the reactant/product is added to the substrate (before the enzyme is added, or vice versa as described herein) at predetermined intervals over a predetermined time period. An advantage of this method is that the addition of the reactant/product to the substrate (in electrolyte) and the addition of the enzyme are performed in the same container using the same electrode.

In at least one embodiment, a customized kit is described containing a solution of enzyme substrate and other necessary reactants in a background electrolyte; a solution of redox active component of enzymatic reaction; and a solution of assayed enzyme. As such, an amperometric measurement is done by using any electrochemical measurement device with amperometric method and a conventional electrochemical cell with the working, reference, and counter electrodes immersed in a solution containing the enzyme substrate. The working electrode is held at a potential E vs. the potential of the reference electrode. The potential E is adequate for either the oxidation or reduction of species present in the solution containing the redox active component of the enzymatic reaction. The experiment is performed by spiking one or more known aliquots of a the redox active containing solution followed by one aliquot of a solution containing assayed enzyme into a stirred solution that contains enzyme substrate and other necessary reactants and measuring the current flowing through the working electrode.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference in their entireties.

Publications disclosed herein are provided solely for their disclosure prior to the filing date of the present invention. Further, the dates of publication provided may be different from the actual publication dates which may need to be independently confirmed.

Each of the applications and patents cited in this text, as well as each document or reference, patent or non-patent literature, cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, as well as each document or reference cited in each of the herein-cited references (including any manufacturer's specifications, instructions, etc.), is hereby expressly incorporated herein by reference.

The following non-limiting examples serve to further illustrate the present disclosure.

### VI. EXAMPLES

### 1. Use of 4-((Tosyl-L-Alanyl)Oxy)Phenyl Tosyl-L-Alaninate in an Internally Calibrated Electrochemical Continuous Enzyme Assay (ICECEA)

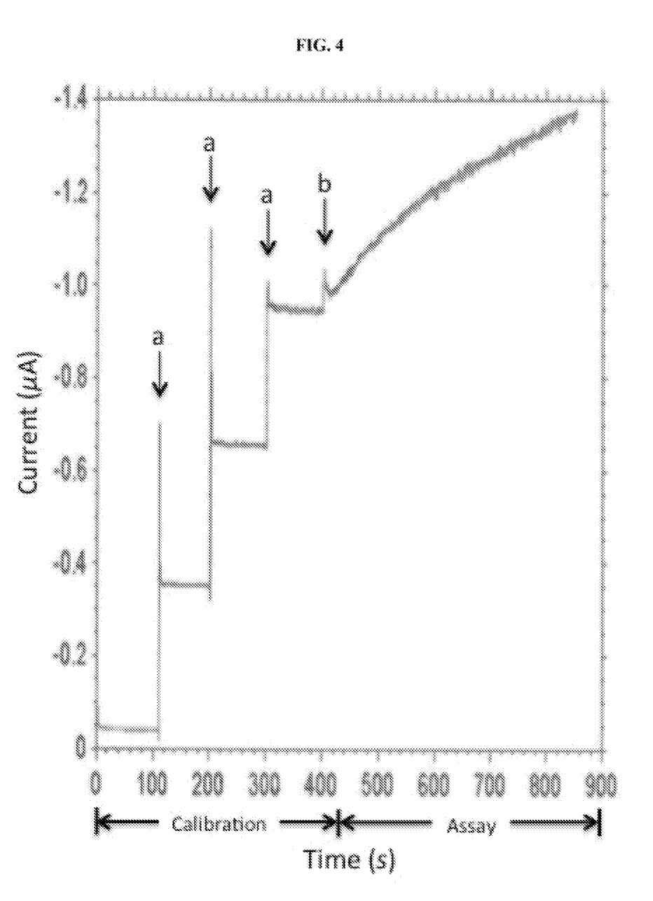

The substrate 4-((tosyl-L-alanyl)oxy)phenyl tosyl-L-alaninate, Compound A below (also referred to as “TAPTA”) was used as a substrate to measure the activity of leukocyte esterase (LE) in an internally calibrated electrochemical continuous enzyme assay (ICECEA). The results are indicated in FIG. 4.

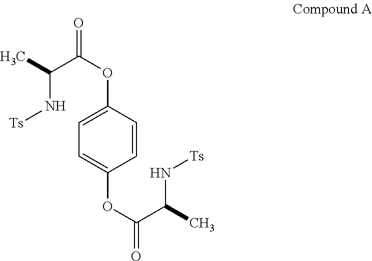 [SMILES: Cc1ccc(S(=O)(=O)N[C@@H](C)C(=O)Oc2ccc(OC(=O)[C@H](C)NS(=O)(=O)c3ccc(C)cc3)cc2)cc1]

The ICECEA was conducted as generally described in U.S. 2016/0040209 as well as in the detailed description supra. Briefly, in the pre-assay phase, three (3) distinct calibration steps were performed by spiking a solution of enzyme substrate (“TAPTA”) and necessary reactants with a solution of the redox active component of the enzymatic reaction. These three distinct calibration steps are denoted by a bold “a” in FIG. 4. After calibration, the assay phase was commenced by spiking one aliquot of assayed enzyme (LE) into the enzyme substrate solution. This step is denoted by a bold “b” in FIG. 4. The enzymatic reaction was followed by measuring current flowing through the working electrode. The enzyme assay was calibrated for LE concentrations ranging from 0-250 μg/L. The enzyme activity of LE demonstrated a linear response relative to LE concentration and predictive of an infection.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following.

What is claimed is:

1. A composition comprising a leukocyte enzyme substrate as depicted in one of Formula I, Formula II, Formula III: 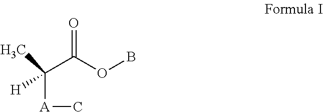
wherein A comprises one of an amino group or an ether group, B is a moiety capable of participating in a redox reaction, and C is an alcohol or amine blocking group; 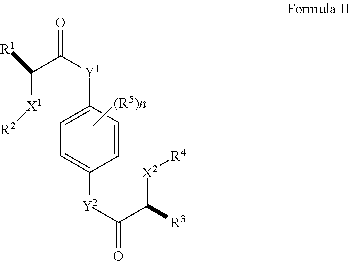
wherein
X¹ and X² are independently O or NR^{a}, and R^{a} is an H, an alkyl or an aryl group;
Y¹ and Y² are independently O or NR^{a};
R¹ and R² are independently an alkyl or an aryl group;
R³ and R⁴ are independently an alkyl, a protecting group or a peptide moiety;
each of the R⁵ on the ring is independently selected from the group consisting of a halogen atom; a hydroxyl group; a C₁-C₆ alkyl group; a C₃-C₆ cycloalkyl group; a C₃-C₆ cycloalkyl C₁-C₆ alkyl group; a C₂-C₆ alkenyl group; a C₂-C₆ alkynyl group; a C₁-C₆ haloalkyl group (including trifluoro C₁-C₆alkyl); a C₂-C₆ haloalkenyl group; a C₂-C₆ haloalkynyl group; a C₃-C₆ halocycloalkyl group; a C₃-C₆ halocycloalkyl C₁-C₆ alkyl group; a C₁-C₆ alkoxy group; a C₃-C₆ cycloalkyloxy group; a C₂-C₆ alkenyloxy group; a C₂-C₆ alkynyloxy group; a C₁-C₆ alkylcarbonyloxy group; a C₁-C₆ haloalkoxy group; a C₁-C₆ alkylthio group; a C₁-C₆ alkylsulfinyl group; a C₁-C₆ alkylsulfonyl group; a C₁-C₆ haloalkylthio group; a C₁-C₆ haloalkylsulfinyl group; a C₁-C₆ haloalkylsulfonyl group; an amino group; a C₁-C₆ alkylcarbonylamino group; a mono(C₁-C₆ alkyl)amino group; a di(C₁-C₆ alkyl)amino group; a hydroxy C₁-C₆ alkyl group; a C₁-C₆ alkoxy C₁-C₆ alkyl group; a C₁-C₆ alkylthio C₁-C₆ alkyl group; a C₁-C₆ alkylsulfinyl C₁-C₆ alkyl group; a C₁-C₆ alkylsulfonyl C₁-C₆ alkyl group; a C₁-C₆ haloalkylthio C₁-C₆ alkyl group; a C₁-C₆ haloalkylsulfinyl C₁-C₆ alkyl group; a C₁-C₆ haloalkylsulfonyl C₁-C₆ alkyl group; a cyano C₁-C₆ alkyl group; a C₁-C₆ alkoxy C₁-C₆ alkoxy group; a C₃-C₆ cycloalkyl C₁-C₆ alkyloxy group; a C₁-C₆ haloalkoxy C₁-C₆ alkoxy group; a cyano C₁-C₆ alkoxy group; a C₁-C₆ acyl group; a C₁-C₆ alkoxyimino C₁-C₆ alkyl group; a carboxyl group; a C₁-C₆ alkoxycarbonyl group; a carbamoyl group; a mono(C₁-C₆ alkyl)aminocarbonyl group; a di(C₁-C₆ alkyl)aminocarbonyl group; a nitro group; and a cyano group; and
n is 0, 1, 2, 3, or 4; and 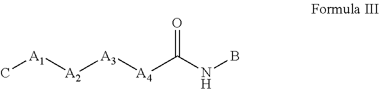
wherein A₁-A₂-As-A₄ represent a core tetrapeptide scaffold sequence which serves as the enzyme active site, B comprises a moiety capable of participating in a redox reaction, and C comprises an acyl group.

2. The composition of claim 1, wherein the leukocyte enzyme substrate comprises Formula I.

3. (canceled)

4. The composition of claim 2, wherein B comprises one of a derivative of phenol, a quinone, a hydroquinone, a substituted quinone, and a substituted hydroquinone.

5-29. (canceled)
